(19) United States
(12) Patent Application Publication
(10) Pub. No.: US 2015/0160206 A1
(43) Pub. Date: Jun. 11, 2015

(54) MICROFLUIDIC DEVICE AND APPARATUS FOR TESTING THE SAME

(71) Applicant: SAMSUNG ELECTRONICS CO., LTD., Suwon-si (KR)

(72) Inventors: Sung Ha PARK, Suwon-si (KR); Sang Bum PARK, Hwaseong-si (KR); Kyung Mi SONG, Suwon-si (KR); Beom Seok LEE, Osan-si (KR); Sang Hyun LEE, Hwaseong-si (KR)

(73) Assignee: SAMSUNG ELECTRONICS CO., LTD., Suwon-si (KR)

(21) Appl. No.: 14/565,898

(22) Filed: Dec. 10, 2014

Related U.S. Application Data
(60) Provisional application No. 61/979,115, filed on Apr. 14, 2014.

(30) Foreign Application Priority Data
Dec. 10, 2013 (KR) 10-2013-0153342

(51) Int. Cl. G01N 33/543; B01L 3/00

(52) U.S. Cl. CPC G01N 33/54366; B01L 3/502715; B01L 2300/0851; B01L 2300/087; B01L 2300/0636; B01L 2300/0803; B01L 2400/0409; B01L 2200/0621

(57) ABSTRACT

Provided is a microfluidic device that is capable of rapidly performing an in vitro diagnosis and being miniaturized. The microfluidic device includes: a platform which includes a sample injection hole through which a sample may be injected; and a chamber which is formed in the platform and in which a first reagent, which includes target antigens that exist in the sample and antibodies that are specifically combined with the target antigens, and a second reagent, which includes an antigen-enzyme conjugant in which antigens that are specifically combined with the antibodies and enzymes are conjugated, are stored.

### CROSS-REFERENCE TO RELATED APPLICATION

This application claims priority from Korean Patent Application No. 10-2013-0153342, filed on Dec. 10, 2013 in the Korean Intellectual Property Office and U.S. Patent Application No. 61/979,115, filed on Apr. 14, 2014 in the United States Patent and Trademark Office, the disclosures of which are incorporated herein by reference in their respective entireties.

### BACKGROUND

1. Field

Exemplary embodiments relate to a microfluidic device that is capable of performing an in vitro diagnosis using a small amount of sample and an apparatus for testing the microfluidic device.

2. Description of the Related Art

In general, an immunity test, a clinical chemistry test, and the like are performed on a patient's sample so as to perform an in vitro diagnosis. Thus, the immunity test and the clinical chemistry test play a very important role in determining a diagnosis, a treatment, and a prognosis of the patient's state.

The in vitro diagnosis is mainly performed in an inspecting room or a laboratory room of a hospital. However, an in vitro diagnosis device recently needs to be miniaturized so as to facilitate performance of the in vitro diagnosis without a limitation in a place.

In addition, a time required for the in vitro diagnosis needs to be minimized so as to rapidly perform the in vitro diagnosis in an emergency situation.

### SUMMARY

Therefore, it is an aspect of one or more exemplary embodiments to provide a microfluidic device that is capable of rapidly performing an in vitro diagnosis and being miniaturized.

Additional aspects of the exemplary embodiments will be set forth in part in the description which follows and, in part, will be obvious from the description, or may be learned by practice of the exemplary embodiments.

In accordance with one aspect of one or more exemplary embodiments, a microfluidic device includes: a platform which includes a sample injection hole through which a sample is injectable; and a chamber which is formed in the platform and which is configured to store a first reagent, which includes target antigens that exist in the sample and antibodies that are specifically combined with the target antigens, and a second reagent, which includes an antigen-enzyme conjugant in which antigens that are specifically combined with the antibodies and enzymes are conjugated.

The target antigens that exist in the sample and the antigen-enzyme conjugant included in the second reagent may be competitively combined with the antibodies included in the first reagent.

At least one from among the first reagent and the second reagent may include a temperament that is specifically combined with the enzymes of the antigen-enzyme conjugant.

The at least one from among the first reagent and the second reagent may further include a chromogen of which a degree of color varies based on an amount of the temperament that is specifically combined with the enzymes of the antigen-enzyme conjugant.

The platform may include a film-shaped upper plate and a film-shaped lower plate, and the chamber may be formed by bonding the upper plate with the lower plate.

The first reagent may be applied onto a first one of the upper plate and the lower plate and then may be dried, and the second reagent may be applied onto the other one of the upper plate and the lower plate and then may be dried.

The microfluidic device may further include a channel that is formed at the platform and which is configured to connect the sample injection hole with the chamber.

The microfluidic device may further include a filter that is disposed at the sample injection hole and which is configured to filter a particular material included in the sample.

The microfluidic device may further include a sample accommodation chamber that is formed at the platform and which is configured to accommodate the sample injected through the sample injection hole.

The platform may be rotatable, and the sample accommodation chamber may be disposed closer to a center of rotation of the platform than the chamber.

The first reagent may be applied at a first position of inner walls of the chamber and then dried, and the second reagent may be applied at a second position of the inner walls of the chamber and then may be dried, wherein the second position is different than the first position.

The first reagent and the second reagent may be stored in the chamber in a solid state.

The microfluidic device may further include a channel configured to connect the chamber with the sample accommodation chamber.

The first reagent and the second reagent may be stored in the chamber in a liquid state, and the chamber may include a barrier wall that separates a first space in which the first reagent is stored from a second space in which the second reagent is stored.

In accordance with another aspect of one or more exemplary embodiments, a microfluidic device includes: a platform which includes a sample injection hole through which a sample is injectable; and a chamber which is formed in the platform and which is configured to store a first reagent, which includes first enzymes that primarily decompose hemoglobin that exists in the sample, and a second reagent, which includes second enzymes that secondarily decompose the decomposed hemoglobin.

The first enzymes that primarily decompose the hemoglobin may be protease-based, and the second enzymes that secondarily decompose the decomposed hemoglobin may be fructosyl-based.

The platform may include a film-shaped upper plate and a film-shaped lower plate, and the chamber may be formed by bonding the upper plate with the lower plate.

The first reagent may be applied onto a first one of the upper plate and the lower plate and then may be dried, and the second reagent may be applied onto the other one of the upper plate and the lower plate and then may be dried.

The microfluidic device may further include a sample accommodation chamber that is formed at the platform and which is configured to accommodate the sample injected through the sample injection hole, wherein the platform may be rotatable, and the sample accommodation chamber may be disposed closer to a center of rotation of the platform than the chamber.

The first reagent may be applied at a first position of inner walls of the chamber and then dried, and the second reagent may be applied at a second position of the inner walls of the chamber and then may be dried, wherein the second position is different from the first position.

The first reagent and the second reagent may be stored in the chamber in a solid state.

The first reagent and the second reagent may be stored in the chamber in a liquid state, and the chamber may include a barrier wall that separates a first space in which the first reagent is stored from a second space in which the second reagent is stored.

In accordance with still another aspect of one or more exemplary embodiments, an apparatus for testing the microfluidic device includes: a detector configured to radiate light having a particular wavelength onto the chamber and to detect light that is transmitted from the chamber or is reflected from the chamber; and a controller configured to determine a change in at least one from among a plurality of optical characteristics from an output signal of the detector and to calculate a respective increase in a concentration of the target antigens which corresponds to an increase in the change in the at least one of the plurality of optical characteristics.

In accordance with yet still another aspect of one or more exemplary embodiments, an apparatus for testing the microfluidic device includes: a detector configured to radiate first light having a first wavelength onto the chamber and to detect second light that is transmitted from the chamber or is reflected from the chamber, and to radiate third light having a second wavelength that is different from the first wavelength onto the chamber and to detect fourth light that is transmitted from the chamber or is reflected from the chamber; and a controller configured to calculate a concentration of hemoglobin that exists in the sample from at least one from among a plurality of optical characteristics of the first light having the first wavelength and to calculate a concentration of glycated hemoglobin that exists in the sample from at least one from among a plurality of optical characteristics of the third light having the second wavelength.

The first wavelength may be a wavelength in a band of 500 nm, and the second wavelength may be a wavelength in a band of 600 nm.

### BRIEF DESCRIPTION OF THE DRAWINGS

These and/or other aspects will become apparent and more readily appreciated from the following description of exemplary embodiments, taken in conjunction with the accompanying drawings of which:

FIGS. 1 and 2 schematically illustrate sides of a chamber included in a microfluidic device, in accordance with an exemplary embodiment;

FIG. 3 schematically illustrates a composition and a reaction principle of a reagent included in the microfluidic device illustrated in FIGS. 1 and 2;

FIGS. 4 and 5 schematically illustrate a degree of reaction of an antigen-enzyme conjugant and a temperament;

FIG. 6 illustrates a structure of BS3 that is an example of a cross-linker;

FIG. 7 schematically illustrates an operation of combining antigens and enzymes using two cross-linkers;

FIG. 8 schematically illustrates a principle of measuring a concentration of whole hemoglobin and a concentration of glycated hemoglobin that are applied to the microfluidic device illustrated in FIGS. 1 and 2;

FIGS. 9 and 10 illustrate one chamber in which a first reagent R1 and a second reagent R2 are solidified, in accordance with an exemplary embodiment;

FIG. 11 illustrates one chamber in which the first reagent R1 and the second reagent R2 are stored in a liquid state, in accordance with an exemplary embodiment;

FIG. 12 illustrates an exterior of a microfluidic device, in accordance with another exemplary embodiment;

FIG. 13 is an exploded perspective view illustrating a structure of a platform on which a test is performed, of the microfluidic device illustrated in FIG. 12;

FIG. 14 is a side view of the microfluidic device of FIG. 12;

FIGS. 15 and 16 illustrate an example of diagnostic items that may be performed in the microfluidic device of FIG. 12;

FIG. 17 is a top plan view of a microfluidic device, in accordance with still another exemplary embodiment;

FIGS. 18 and 19 illustrate a structure of a chamber included in the microfluidic device illustrated in FIG. 17;

FIGS. 20 and 21 schematically illustrate a reaction that occurs in a sample injected into the microfluidic device of FIGS. 1 and 2;

FIG. 22 illustrates an exterior of an apparatus for testing the microfluidic device of FIG. 12;

FIG. 23 illustrates an exterior of an apparatus for testing the microfluidic device of FIG. 17;

FIG. 24 illustrates movement of a sample within the microfluidic device mounted on the apparatus for testing the microfluidic device;

FIG. 25 is a graph showing an optical density (OD) obtained by radiating light having a wavelength of 630 nm onto a chamber in which a reagent for detecting glycated hemoglobin is stored;

FIG. 26 is a graph showing an OD obtained by radiating light having a wavelength of 535 nm onto the same chamber;

FIG. 27 is a graph showing a result of measuring OD by varying a concentration of enzymes included in the reagent with respect to a sample including glycated hemoglobin having the same concentrations;

FIG. 28 is a graph showing a result of measuring OD by increasing a concentration of enzymes by a factor of ten with respect to a sample having glycated hemoglobin having different concentrations;

FIG. 29 is a graph showing linearity of a result of testing by a microfluidic device, in accordance with an exemplary embodiment; and

FIG. 30 is a graph showing correlation of a result of testing a microfluidic device, in accordance with an exemplary embodiment.

### DETAILED DESCRIPTION

Reference will now be made in detail to exemplary embodiments, examples of which are illustrated in the accompanying drawings, wherein like reference numerals refer to like elements throughout.

Since an immunity test, a clinical chemistry test, and the like are used in an in vitro diagnosis, an immunity method, in which reactions, such as enzyme-linked immunospecific assay (ELISA), difference gel electrophoresis (DIGE), and the like, occur stepwise, is applied to the immunity test.

In detail, steps of the immunity test to which ELISA is applied, will be briefly described. First, a reagent which includes primary antibodies is added to a blood sample which includes target antigens, so that the target antigens and the primary antibodies can be specifically combined with each other. Materials that are non-specifically combined are removed by a washing process.

Secondary antibodies labeled with enzymes, such as horseradish peroxidase (HRP) or alkaline phosphatase (AP), are added to the blood sample that reacts with the primary antibodies so that the primary antibodies and the secondary antibodies can be specifically combined with each other. Then, the non-specifically-combined materials can be removed by washing, and a change in color due to an enzyme reaction can be measured so that a concentration of the target antigens can be estimated.

Each of the steps of the reaction ELISA requires at least three minutes, and the steps are sequentially performed, and a total test time is approximately at least twenty minutes. This causes a disturbance in rapidly performing the in vitro diagnosis with respect to a patient's sample in an emergency situation. In addition, a space in which a reaction may occur is required in each step. However, this causes a disturbance in performing miniaturization of an in vitro diagnosis device.

Thus, in a microfluidic device in accordance with an exemplary embodiment, a plurality of reactions that constitute the immunity test or clinical chemistry test occur in one chamber, so that miniaturization of the in vitro diagnosis device and rapidity of performing a test can be implemented. Hereinafter, detailed exemplary embodiments of the microfluidic device will be described.

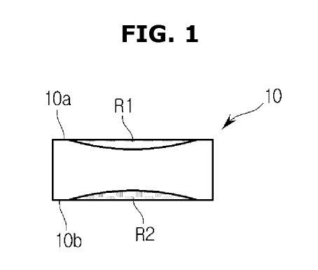

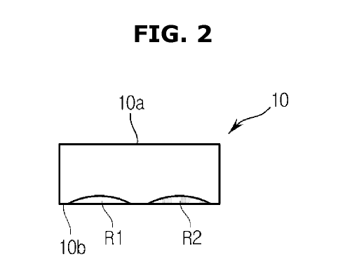

FIGS. 1 and 2 schematically illustrate sides of a chamber included in a microfluidic device, in accordance with an exemplary embodiment.

The microfluidic device is a device that performs the in vitro diagnosis using a small amount of sample. The chamber is a predetermined space which is formed in the microfluidic device and in which a sample or reagent is accommodated or a reaction of the sample or reagent occurs. A detailed exemplary embodiment of a structure of the microfluidic device will be described below.

When two reagents, i.e., a first reagent R1 and a second reagent R2, are used in the immunity test or clinical chemistry test for the in vitro diagnosis, the first reagent R1 and the second reagent R2 may be respectively solidified at inner walls 10a and 10b of a chamber 10 that face each other, as illustrated in FIG. 1, or the first reagent R1 and the second reagent R2 may be solidified at the same inner wall 10b, as illustrated in FIG. 2. However, solidification is not essential to the immunity test or clinical chemistry test for the in vitro diagnosis, and a liquid reagent may be applied onto the inner wall and then may not undergo a drying procedure. Further, the first reagent R1 and the reagent R2 may be accommodated in the chamber 10 in a liquid state without being solidified. However, an exemplary embodiment thereof will be described below.

When the sample is injected into the chamber 10, the sample reacts with the first reagent R1 and the second reagent R2, and a result of reaction is measured so that a concentration of a target material can be estimated. In this manner, the in vitro diagnosis can be completed in one step.

Hereinafter, exemplary embodiments of a testing method that may be applied to the microfluidic device illustrated in FIGS. 1 and 2, and a composition of a reagent will be described.

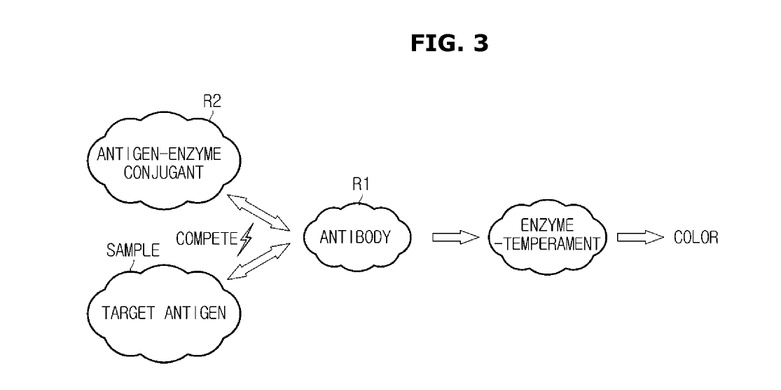

FIG. 3 schematically illustrates a composition and a reaction principle of a reagent included in the microfluidic device illustrated in FIGS. 1 and 2, and FIGS. 4 and 5 schematically illustrate a degree of reaction of an antigen-enzyme conjugant and a temperament.

Referring to FIG. 3, the first reagent R1 includes antibodies, and the second reagent R2 includes an antigen-enzyme conjugant in which antigens and enzymes are conjugated. In particular, the antigens included in the antigen-enzyme conjugant are antigens that are specifically combined with the antibodies included in the first reagent R1, and the antibodies included in the first reagent R1 are antibodies that are specifically combined with target antigens included in the sample. Thus, the antigens included in the antigen-enzyme conjugant may be antigens of the same type as the target antigens.

When the sample is injected into the chamber 10, a first reaction caused by the first reagent R1 and a second reaction caused by the second reagent R2 occur simultaneously. Since the antigens of the antigen-enzyme conjugant and the target antigens included in the sample are specifically combined with the antibodies included in the first reagent R1, a reaction of the antigens of the antigen-enzyme conjugant and the antibodies, and a reaction of the target antigens and the antibodies correspond to competition reactions.

Further, since the antigens of the antigen-enzyme conjugant are artificial products and the target antigens included in the sample exist in a human body, reactivity of the target antigens and the antibodies is superior to reactivity of the antigens of the antigen-enzyme conjugant and the antibodies. Thus, the more the target antigens react with the antibodies included in the first reagent R1, the greater the reduction of the number of the antibodies to be combined with the antigens of the antigen-enzyme conjugant. Thus, the concentration of the target antigens included in the sample can be estimated from an amount of the antigen-enzyme conjugant which is combined with the antibodies.

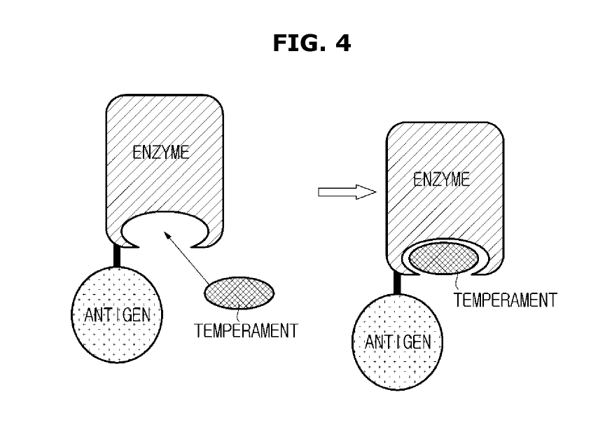

Referring to FIG. 4, in a state in which the antigen-enzyme conjugant is not combined with the antibodies, a temperament that is specifically combined with enzymes is specifically combined with the enzymes of the antigen-enzyme conjugant, thereby forming an enzyme-temperament complex. The temperament that is specifically combined with the enzymes is included in the first reagent R1 or the second reagent R2.

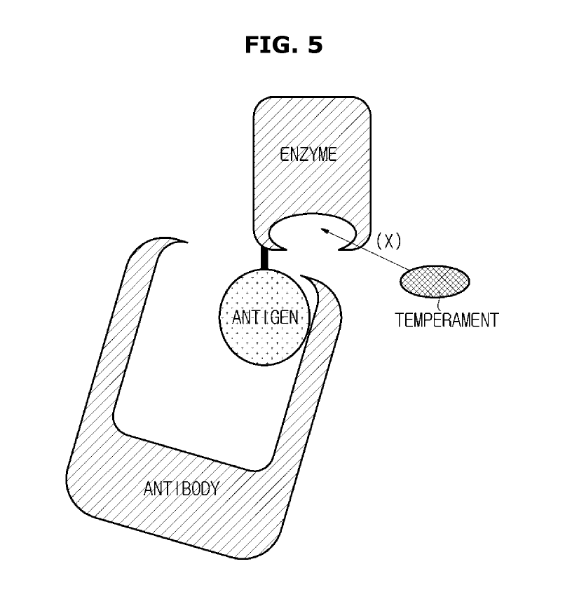

Referring to FIG. 5, in a state in which the antigen-enzyme conjugant is combined with the antibodies, the temperament that is specifically combined with the enzymes is disturbed by the antibodies and does not enter an active site of the enzymes. Thus, the antigen-enzyme conjugant combined with the antibodies is not combined with the temperament.

Materials used in a color reaction are included in the first reagent R1 or second reagent R2, and the enzyme-temperament complex affects the color reaction. Thus, a degree of the color reaction varies according to an amount at which the antigen-enzyme conjugant is combined with the antibodies, and as such, a change in one or more optical characteristics caused by the color reaction can be measured so that the concentration of the target antigens included in the sample can be estimated. In particular, the optical characteristics may include one or more from among optical density (OD), reflectance, luminous efficiency, and transmittance with respect to light having a particular wavelength.

As described above, when the sample and the reagent react with each other, there may be a material that is non-specifically combined with the antigens or antibodies, as well as a specific combination of the antigens and the antibodies. In an existing immunity test, the non-specific combination affects the result of estimating the concentration of the target antigens so that a washing procedure is required to remove the material that is non-specifically combined with the antigens or antibodies. However, if the concentration of the target antigens is estimated using the competition reactions of the antigen-enzyme conjugant and the target antigens, as described above with reference to FIGS. 3, 4, and 5, the non-specific combination does not affect the result of estimating the target antigens. Thus, the washing procedure for removing the material that is non-specifically combined with the antigens or antibodies may be omitted. Thus, an additional washing chamber is not required so that a reaction can be finished in one step using one chamber 10.

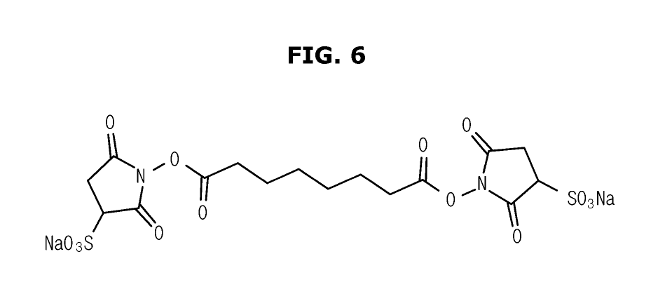

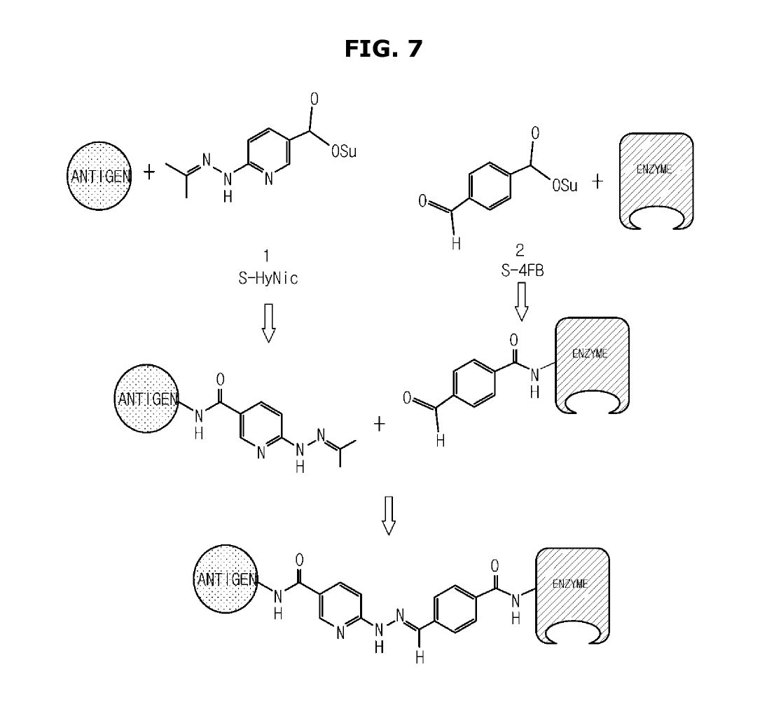

FIG. 6 illustrates a structure of BS3 that is an example of a cross-linker, and FIG. 7 schematically illustrates an operation of combining antigens and enzymes using two cross-linkers.

A cross-linker may be used to create the antigen-enzyme conjugant included in the first reagent R1. Any type of a cross-linker via which antigens and enzymes may be conjugated may be used. For example, a Bis[sulfosuccinimidyl] suberate (BS3) having a molecular structure as illustrated in FIG. 6 may be used. BS3 may connect amine groups (−NH3). Thus, if the antigens and the enzymes react with BS3, the antigens are combined with one end of the BS3 molecule, and the enzymes are combined with the other end of the BS3 molecule, so that the antigen-enzyme conjugant can be created.

As another example, two cross-linkers, such as Sulfo-Succinimidyl-6-Hydrazino-Nicotinamide (Sulfo-S-HyNic) and Sulfo-Succinimidyl-4-FormylBenzamide (Sulfo-S-4FB), may be used, as illustrated in FIG. 7.

Sulfo-S-HyNic is combined with one biomolecule, such as protein, oligos, or peptides via primary amine, and Sulfo-S-4FB is combined with two biomolecules, such as protein, oligos, or peptides via primary amine.

Referring to FIG. 7, the antigens are combined with one end of the Sulfo-S-HyNic molecule, and the enzymes are combined with one end of the Sulfo-S-4FB molecule. Sulfo-S-HyNic, with which the antigens are combined, and Sulfo-S-4FB, with which the enzymes are combined, are combined with each other such that the antigen-enzyme conjugant, in which the antigens and the enzymes are combined, is created.

A method of estimating the concentration of the target material may be applied to all items of the immunity test that uses an antigen-antibody reaction, as described above. Hereinafter, as a detailed example, an example in which the method of estimating the concentration of the target material is applied to thyroid-stimulating hormone (TSH) from among items of the immunity test, will be described.

In a state in which a G3PDH-TSH antigen conjugant, which is created by combining a G3PDH enzyme with a TSH antigen using a cross-linker, is not combined with anti-TSH, the G3PDH-TSH enzyme is specifically combined with Glycerol-3-phosphate that is a temperament so that an enzyme-temperament complex can be formed, and if NAD is added to the enzyme-temperament complex, Dihydroxyacetone-3-phosphate and NADH are created, as shown in the following Formula 1.

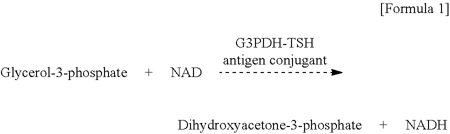

NADH that is created by applying Formula 1 above reacts with WST-4(2-Benzothiazolyl-3-(4-carboxy-2-methoxyphenyl)-5-[4-(2-sulfoethylcarbamoyl)phenyl]-2H-tetrazolium that is a chromogen in the presence of diaphorase that serves as a catalyst, so that Formazan and NAD are created, as shown in the following Formula 2.

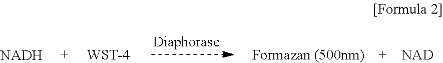

Formazan is a pigment that represents blue or violet, and OD may be measured by using Formazan at a wavelength of about 550 nm. Thus, when the above-described reaction principle is used, light having a wavelength of 540 nm to 560 nm is radiated onto the chamber 10, and light that is transmitted from or is reflected from the chamber 10 is detected so that OD can be measured, and a concentration of the TSH antigen can be estimated based on the measured OD.

When the reaction principle is used, an example of a composition of a required reagent is shown in the following Table 1.

TABLE 1

| First reagent R1 | Second reagent R2 |
|---|---|
| anti-TSH | G3PDH-TSH antigen conjugant |
| WST-4 | Glycero-3-phosphate |
| NAD | Diaphorase |
| KCl | Bicine |
| MgCl2 | MgCl2 |
| MES | |

All of Bicine, KCl, MgCl2, and MES are buffers. In addition, chaps, sugar alcoholic-based sorbitol, mannitol, or Trehalose may be added for stability of the enzymes, the antigens, and the antibodies included in the first reagent and the second reagent, and EDTA 2Na, Ascorbic acid, DL-Dithiothreitol, BSA, Ethylene glycol, Glycerol, β-mercaptoethanol, and Ethylene glycol may be further added for stability of the color reaction.

However, the above-described composition of the reagent is merely an example that may be applied to diagnose TSH in an exemplary embodiment, and the composition of the reagent may be different from the above Table 1 as required. In detail, other enzymes than Glycerol-3-phosphate may be used as an enzyme of the antigen-enzyme conjugant, and types of a chromogen and a buffer may vary according to reaction products that vary according to the enzymes. Alternatively, a change in color that appears due to enzyme reaction products that do not include the chromogen may also be measured. Further, the composition of the reagent may vary according to an item of the immunity test to be performed.

In the above-mentioned manner, if the first reagent R1, which includes the antibodies, and the second reagent R2, which includes the antigen-enzyme conjugant, are stored in one chamber 10, not several steps but one step is undergone so as to detect the target antigens so that an existing immunity test time that typically takes 20 minutes or more can be greatly reduced to one minute. In addition, a diagnosis can be performed by using only one chamber, without including a plurality of chambers that are required to perform several steps. Thus, miniaturization of the microfluidic device can be implemented. A description of a structure of the microfluidic device will be described below.

The microfluidic device illustrated in FIGS. 1 and 2 may also be applied to the in vitro diagnosis that uses the clinical chemistry test, in particular, to a test that measures a concentration of glycated hemoglobin (HbA1c) from among procedures of the clinical chemistry test. HbA1c is created by combining hemoglobin molecules of red blood cells that transport oxygen with glucose in the blood. A count of HbA1c may be recognized as an indicator which corresponds to a blood sugar amount for two to three months.

Since a quantitative count of HbA1c is represented as a ratio, a concentration of total hemoglobin in the blood is also measured so as to obtain a count of HbA1c and is represented as % HbA1c[HbA1c/whole hemoglobin]. Since a concentration of whole hemoglobin and a concentration of HbA1c are individually measured in the related art, a relatively long time is required in order to perform detection while executing a reagent reaction in several steps, and a plurality of chambers must be provided at the microfluidic device so as to perform several steps.

Referring back to FIGS. 1 and 2, with respect to the microfluidic device illustrated in FIGS. 1 and 2, both the first reagent R1 and the second reagent R2 may be provided at one chamber 10 so that the sample can react with the first reagent R1 and the second reagent R2 simultaneously in one chamber 10. In the example, the first reagent R1 and the second reagent R2 are reagents used to measure the concentration of whole hemoglobin and the concentration of HbA1c.

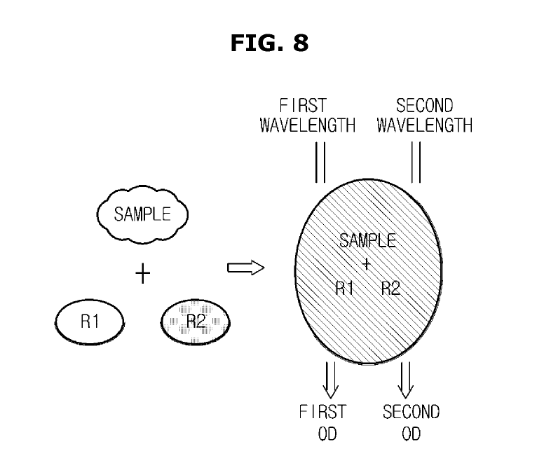

FIG. 8 schematically illustrates a principle of measuring a concentration of whole hemoglobin and a concentration of glycated hemoglobin that are applied to the microfluidic device illustrated in FIGS. 1 and 2.

Since both the first reagent R1 and the second reagent R2 are stored in one chamber 10, as illustrated in FIGS. 1 and 2, if the sample is injected into the chamber 10, the sample may react with the first reagent R1 and the second reagent R2 simultaneously, as illustrated in FIG. 8.

Light having a first wavelength may be radiated onto the chamber 10 in which a reaction between the sample and the first reagent R1 and the second reagent R2 occurs, so that first OD can be measured, and light having a second wavelength may be radiated onto the chamber 10 so that second OD can be measured. The first wavelength may be in a band of 500 nm, for example, 570 nm, and the concentration of whole hemoglobin can be estimated from the first OD. The second wavelength may be in a band of 600 nm, for example, 660 nm, and the concentration of glycated hemoglobin can be estimated from the second OD.

Compositions of the first reagent R1 and the second reagent R2 for detecting HbA1c are shown in the following Table 2. Thus, a more detailed exemplary embodiment will now be described with reference to the following reagent compositions.

TABLE 2

| First reagent R1 | Second reagent R2 |
|---|---|
| protease | FPOX |
| chromogen | POD |

Referring to Table 2, a protease-based enzyme and a chromogen are included in the first reagent R1, and fructosyl peptide oxidase (FPDX) that is a fructosyl-based enzyme and peroxidase (POD) are included in the second reagent R2. Other fructosyl-based enzymes other than FPDX may also be used in the second reagent R2.

Further, although not shown in Table 2, buffers may be further included in either or both of the first reagent R1 and the second reagent R2, respectively. In addition, chaps, sugar alcoholic-based sorbitol, mannitol, or Trehalose may be added for stability of the enzymes, the antigens, or the antibodies included in the first reagent R1 and the second reagent R2, and EDTA 2Na, Ascorbic acid, DL-Dithiothreitol, BSA, Ethylene glycol, Glycerol, β-mercaptoethanol, and Ethylene glycol may be further added for stability of the color reaction.

The sample is injected into the chamber 10 in which the first reagent R1 and the second reagent R2 having the compositions of Table 2 are stored, and light having a wavelength of a 500 nm band, for example, 570 nm, is radiated onto the chamber 10 so that OD can be measured. The concentration of hemoglobin can be estimated from the measured OD. In particular, the sample may be whole blood, i.e., whole blood in which a hemolyzing solution is added and hemoglobin is separated from the red blood cells. For example, the hemolyzing solution may be a lysis buffer that includes a surfactant.

Fructosylated dipeptides which include an N-end of a β-chain of hemoglobin are separated (primarily separated) or decomposed by protease included in the first reagent R1. If oxidative cleaving (secondary separation) of fructosylated dipeptides occurs due to FPDX included in the second reagent R2, hydrogen peroxide (H₂O₂) is created, and the created H₂O₂ reacts with POD and an appropriate chromogen and represents color.

In particular, the first reagent R1 may include an enzyme that primarily decomposes hemoglobin that exists in the sample, and the second reagent R2 may include an enzyme that secondarily decomposes decomposed hemoglobin. Thus, light having a wavelength of a 600 nm band may be radiated onto the chamber 10 so that OD can be measured, and the concentration of HbA1c can be measured from measured OD.

The composition of the reagent of Table 2 is also merely an example that may be applied to FIGS. 1 and 2, and the composition of the reagent may be different from Table 2 as required.

Hereinafter, various exemplary embodiments of the above-described chamber and the structure of the microfluidic device will be described in detail.

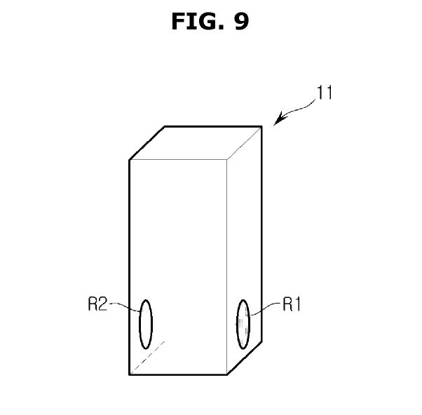

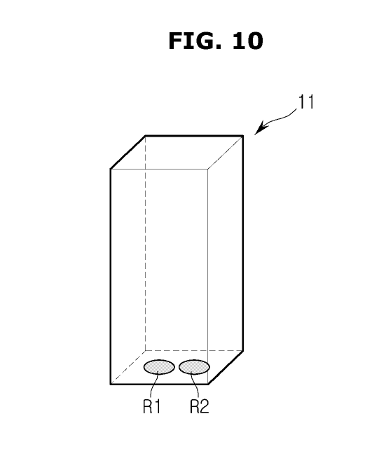

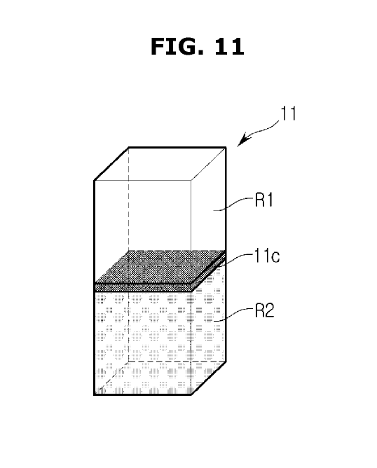

FIGS. 9 and 10 illustrate one chamber in which a first reagent R1 and a second reagent R2 are solidified, in accordance with an exemplary embodiment, and FIG. 11 illustrates one chamber in which the first reagent R1 and the second reagent R2 are stored in a liquid state, in accordance with an exemplary embodiment.

As a detailed exemplary embodiment of the chamber 10 illustrated in FIGS. 1 and 2, a cuvette-shaped chamber 11 as illustrated in FIGS. 9, 10, and 11 may be used, and the chamber 11 itself may be the microfluidic device illustrated in FIGS. 1 and 2. When the chamber 11 itself is the microfluidic device, a housing that constitutes the chamber 11 is a platform of the microfluidic device.

The first reagent R1 and the second reagent R2 may be applied and then dried at facing inner walls, as illustrated in FIG. 9, or may be separated from each other, applied, and then dried at the same inner wall, as illustrated in FIG. 10. In particular, the first reagent R1 and the second reagent R2 may be solidified at the inner walls of the chamber 11 or may be solidified in a separated state for stability of the reagent. However, drying after applying of the first reagent R1 and the second reagent R2 is not essential, and the first reagent R1 and the second reagent R2 may also be in a liquid state without being solidified.

Alternatively, the first reagent R1 and the second reagent R2 may also be applied to adjacent inner walls, instead of facing inner walls. Thus, only if the first reagent R1 and the second reagent R2 are separated from each other at the inner walls of the chamber 11, positions in which the first reagent R1 and the second reagent R2 are solidified, are not limited.

As illustrated in FIG. 11, the first reagent R1 and the second reagent R2 may also be stored in a liquid state in the chamber 11. In this case, in order to prevent the first reagent R1 and the second reagent R2 from reacting with each other, a barrier wall 11c may be installed in an inner space of the chamber 11 so that a first space in which the first reagent R1 is stored and a second space in which the second reagent R2 is stored can be separated from each other via the barrier wall 11c.

As described above, the chamber 11 itself may be the microfluidic device. Thus, the first reagent R1 and the second reagent R2 are stored in the chamber 11, and the sample is injected into the chamber 11 in which the first reagent R1 and the second reagent R2 are stored. If the chamber 11 into which the sample is injected, is inserted into a spectrometer, the spectrometer may radiate light having a predetermined wavelength onto the chamber 11, and radiated light may be detected so that one or more optical characteristics of a reaction resultant within the chamber 11 or a change in the one or more optical characteristics which change is caused by the reaction resultant can be measured. The sample injected into the chamber 11 may be a biosample, such as blood, a tissue liquid, a lymph liquid, urine, or the like, or a sample that is pre-treated, such as centrifugation, dilution, or hemolysis.

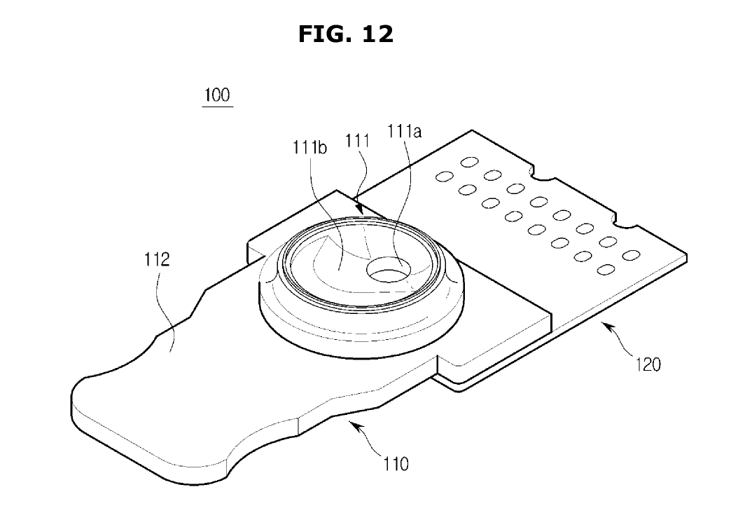

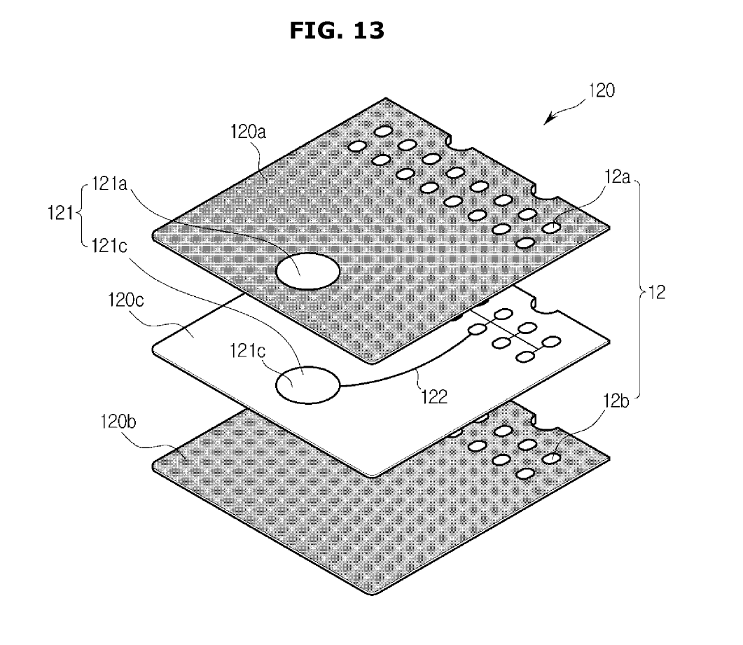

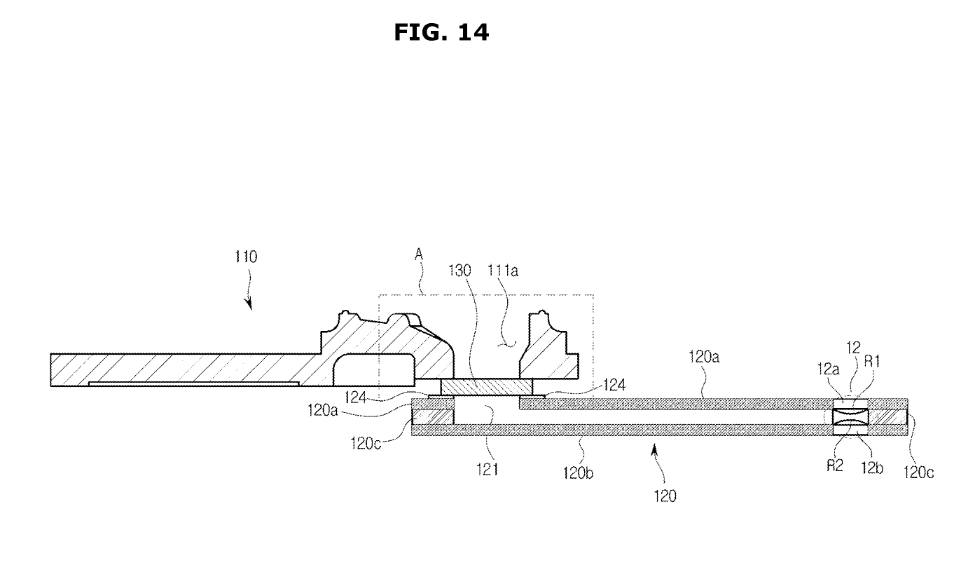

FIG. 12 illustrates an exterior of a microfluidic device in accordance with another exemplary embodiment, FIG. 13 is an exploded perspective view illustrating a structure of a platform, on which a test is performed, of the microfluidic device illustrated in FIG. 12, and FIG. 14 is a side view of the microfluidic device of FIG. 12.

Referring to FIG. 12, a microfluidic device 100 in accordance with another exemplary embodiment may include a housing 110 and a film-shaped platform 120 in which a sample and a reagent meet each other, and in which a reaction thereof occurs.

The housing 110 may support the platform 120 and simultaneously may cause a user to hold the microfluidic device 100. The housing 110 may be formed of a material that is easily formed and that is chemically and biologically inactive.

For example, one of various materials, such as a plastic material, for example, acryl, such as polymethylmethacrylate (PMMA), polysiloxane, such as polydimethylsiloxane (PDMS), polycarbonate (PC), polyethylene, for example, linear low density polyethylene (LLDPE), low density polyethylene (LDPE), medium density polyethylene (MDPE), or high density polyethylene (HDPE), polyvinylalcohol (PVA), very low density polyethylene (VLDPE), polypropylene (PP), acrylonitrile butadien styrene (ABS), cyclo olefin copolymer (COC), glass, mica, silica, or a semiconductor wafer, may be used to form the housing 110.

A sample supply portion 111, to which the sample is supplied, is provided at the housing 110. The sample supplied to the microfluidic device 100 may include a biosample, such as blood, a tissue liquid, a lymph liquid, urine, or the like. The sample supply portion 111 includes a supply hole 111a through which the supplied sample flows into the platform 120, and a supply assisting portion 111b that assists with supply of the sample.

The user may drop the sample to be tested by using a tool, such as a pipette or eyedropper, to drop the sample into the supply hole 111a, and the supply assisting portion 111b that is formed to be inclined at a periphery of the supply hole 111a in a direction of the supply hole 111a may cause a fluid sample that has dropped into the periphery of the supply hole 111a to flow into the supply hole 111a.

The platform 120 may be bonded to a lower part of the sample supply portion 111 of the housing 110, or may be combined with the housing 110 while being inserted into a predetermined groove formed in the housing 110.

Referring to FIG. 13, the platform 120 may have a structure in which three plates 120a, 120b, and 120c are bonded together. Three plates may be divided into an upper plate 120a, a lower plate 120b, and a middle plate 120c. The upper plate 120a and the lower plate 120b that are printed with a shielding ink may protect the sample that is moved to a chamber 12 from external light.

The upper plate 120a and the lower plate 120b may be formed of films, and the films used to form the upper plate 120a and the lower plate 120b may be one selected from among a polyethylene film, such as VLDPE, LLDPE, LDPE, MDPE, or HDPE, a PP film, a polyvinyl chloride (PVC) film, a PVA film, a polystyrene (PS) film, and a polyethylene terephthalate (PET) film.

The middle plate 120c of the platform 120 may be formed of a porous sheet, such as cellulose, and the middle plate 120c itself may serve as a vent, and the porous sheet may be formed of a material having hydrophobicity, or hydrophobic treatment may be performed on the porous sheet, so that the porous sheet may not affect movement of the sample.

A channel 122, via which the sample is injected through a sample injection hole 121 is moved to the chamber 12, and the chamber 12, in which a reaction of the sample and the reagent occurs, are formed at the platform 120. When the platform 120 has a triple layer structure, an upper plate hole 121a that constitutes the sample injection hole 121 may be formed in the upper plate 120a, and a portion 12a corresponding to the chamber 12 may be processed transparently.

Further, a portion 12b of the lower plate 120b that corresponds to the chamber 12 may also be processed transparently. Thus, the portions 12a and 12b that correspond to the chamber 12 are transparently processed so that optical characteristics caused by the reaction that occurs in the chamber 12 can be measured.

A middle plate hole 121c that constitutes the sample injection hole 121 is formed in the middle plate 120c, and if the upper plate 120a, the middle plate 120c, and the lower plate 120b are bonded together, the upper plate hole 121a and the middle plate hole 121c overlap each other so that the sample injection hole 121 of the platform 120 can be formed.

Since the chamber 12 is formed in an opposite region to the middle plate hole 121c from among regions of the middle plate 120c, a region that corresponds to the chamber 12 from among the regions of the middle plate 120c may be removed in a predetermined shape, such as a circular shape or a rectangular shape, and the upper plate 120a, the middle plate 120c, and the lower plate 120b may be bonded together so that a reagent chamber 12 can be formed.

In addition, the channel 122 having a width of 1 to 500 μm is formed at the middle plate 120c, so that the sample injected through the sample injection hole 121 can be moved up to the chamber 12 due to a capillary force of the channel 122. However, the width of the channel 122 is merely an example that may be applied to the microfluidic device 100, and exemplary embodiments are not limited thereto.

Referring to FIG. 14, the sample supplied through the supply hole 111a flows into the platform 120 via the sample injection hole 121 formed in the platform 120. Thus, a filter 130 may be disposed between the sample supply portion 111 and the sample injection hole 121 so as to filter the sample supplied to the sample supply portion 111, and the filter 130 and the platform 120 may be adhered to each other via an adhesive 124.

The filter 130 may be implemented as a porous polymer membrane, such as PC, polyethersulfone (PES), PE, polysulfone (PS), or polyarylsulfone (PASF). When a blood sample is supplied, blood passes through the filter 130, and a blood cell may be filtered by the filter 130, and only blood plasma or serum may flow into the platform 120.

The first reagent R1 and the second reagent R2 may be stored in the chamber 12. For example, the first reagent R1 may be applied onto an upper inner wall 12a of the chamber 12, and the second reagent R2 may be applied onto a lower inner wall 12b of the chamber 12 and then may be dried. Thus, positions of the first reagent R1 and the second reagent R2 may be reversed. In particular, the upper inner wall 12a and the lower inner wall 12b of the chamber 12 are the portion corresponding to the chamber 12 of the upper plate 120a and the portion corresponding to the chamber 12 of the lower plate 120b, respectively.

If the sample is supplied to the sample supply portion 111 of the microfluidic device 100, the supplied sample flows into the platform 120 via the sample injection hole 121, and the flowed sample is moved to the chamber 12 via the channel 122. The sample that is moved to the chamber 12 reacts with the first reagent R1 and the second reagent R2 stored in the chamber 12 simultaneously and causes optical characteristics used to estimate a concentration of target antigens that exist in the sample or a concentration of whole hemoglobin and a concentration of HbA1c that exist in the sample, or a change in the optical characteristics.

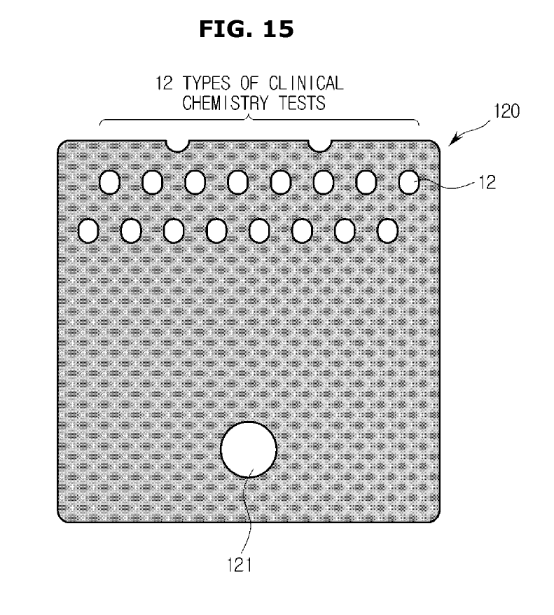

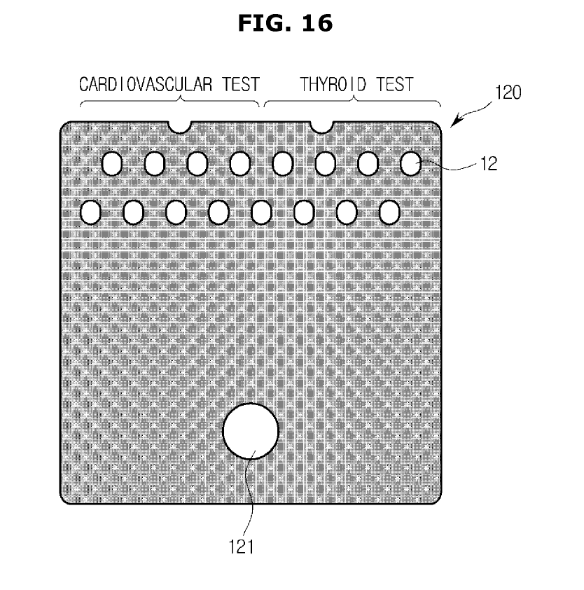

FIGS. 15 and 16 illustrate an example of diagnostic items that may be performed in the microfluidic device of FIG. 12.

In the microfluidic device 100, because a test may be completed in one chamber 12, several tests can be simultaneously performed in one microfluidic device 100. For example, twelve types of clinical chemistry tests can be performed using one microfluidic device 100, as illustrated in FIG. 15. To this end, the first reagent R1 and the second reagent R2 that are required to perform clinical chemistry tests are stored in a plurality of chambers 12 provided at the platform 120. Compositions of the first reagent R1 and the second reagent R2 may vary according to types of the clinical chemistry tests performed in each of the plurality of chambers 12.

Alternatively, different types of immunity tests may also be performed using one microfluidic device 100. For example, a cardiovascular test may be performed in a first part of the plurality of chambers 12, and a thyroid test may be performed in a second part of the plurality of chambers 12, as illustrated in FIG. 16. To this end, the first reagent R1 and the second reagent R2 that are required to perform the cardiovascular test and the first reagent R1 and the second reagent R2 that are required to perform the thyroid test are stored in each of the plurality of chambers 12.

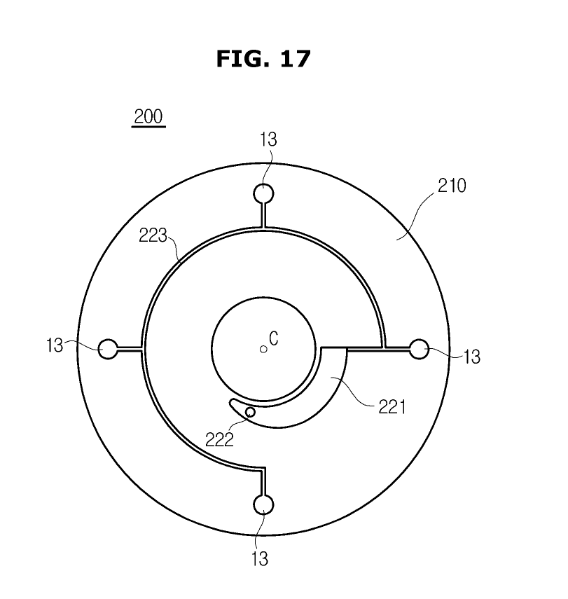

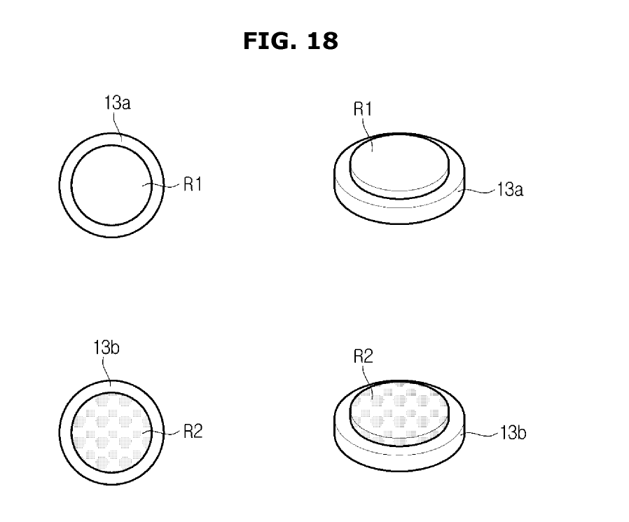

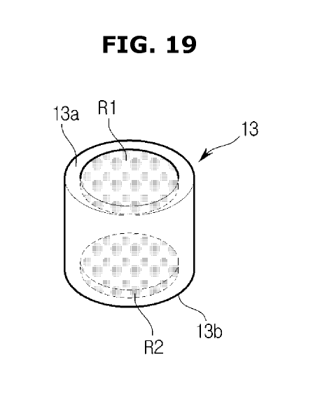

FIG. 17 is a top plan view of a microfluidic device, in accordance with still another exemplary embodiment, and FIGS. 18 and 19 illustrate a structure of a chamber included in the microfluidic device illustrated in FIG. 17.

Referring to FIG. 17, a microfluidic device 200 in accordance with still another exemplary embodiment may include a rotatable platform 210 and a plurality of microfluidic structures which are formed in the platform 210.

Each of the microfluidic structures includes a plurality of chambers in which a sample or reagent is accommodated, and a channel that connects the plurality of chambers. The microfluidic structures are formed in the microfluidic device 200. However, in the current exemplary embodiment, the microfluidic device 200 is formed of a transparent material, and when the microfluidic device 200 is viewed from above, microfluidic structures formed in the microfluidic device 200 may be seen.

The platform 210 may be formed of a material which is easily formed and of which surface is biologically inactive. For example, the platform 210 may be formed of one of various materials, such as a plastic material, for example, PMMA, PDMS, PC, PP, PVA, or PE, glass, mica, silica, and a silicon wafer.

However, exemplary embodiments are not limited thereto. Any type of material having chemical and biological stability and mechanical processibility may serve as the material of the platform 210, and when a test result within the microfluidic device 200 is optically analyzed, the platform 210 may further have optical transparency.

The microfluidic device 200 may move materials within the microfluidic structures by using a centrifugal force caused by rotation. In FIG. 17, a disk-shaped platform 210 is shown. However, the platform 210 used in the current exemplary embodiment may have a fan shape, as well as a full disk shape, or a polygonal shape that may be rotatable.

In the current exemplary embodiment, the microfluidic structures may not be structures having a particular shape, but instead may be structures, such as chambers or channels formed on the platform 210, or may also be comprehensive materials that perform particular functions as needed. The microfluidic structures may perform different functions according to respective characteristics of arrangement or respective types of accommodated materials.

The platform 210 includes a sample injection hole 222, a sample accommodation chamber 221 in which a sample injected into the sample injection hole 222 is accommodated and is supplied to another chamber, a chamber 13 in which the first reagent R1 and the second reagent R2 are stored, and a distribution channel 223 that distributes the sample accommodated in the sample accommodation chamber 221 into the chamber 13. Further, although not shown, when blood is used as the sample, a microfluidic structure for centrifugal separation of blood may be further provided in the microfluidic device 200 as required, and a metering chamber for moving a quantitative sample to the chamber 13, and a buffer chamber in which a buffer liquid is accommodated, may be additionally provided.

The platform 210 may include a plate having a plurality of layers. For example, when the platform 210 includes two plates, i.e., an upper plate and a lower plate, an intagliated structure which corresponds to the microfluidic structure, such as a chamber, is formed on a surface on which the upper plate and the lower plate contact each other, and the two plates are bonded to each other so that a space in which a fluid may be accommodated and a path along which the fluid may move can be formed in the platform 210. Bonding of the plates may be performed using any one of various methods, such as adhesion using an adhesive or a double-sided adhesive tape, ultrasonic fusion, or laser welding.

In order to store the first reagent R1 and the second reagent R2 in the chamber 13, the first reagent R1 and the second reagent R2 may be applied onto a portion in which the intagliated structure which corresponds to a reagent chamber 224 of the upper plate or lower plate of the platform 210 is formed, and then may be dried. Further, the upper plate and the lower plate may be bonded to each other.

When only the chamber 13 is separated from the microfluidic device 200, the first reagent R1 may be applied onto inner walls of an upper surface 13a of the chamber 13 and then may be dried, and the second reagent R2 may be applied onto inner walls of the lower surface 13b and then may be dried, and the upper surface 13a and the lower surface 13b can be bonded to each other so that one chamber 13 can be formed, as illustrated in FIGS. 18 and 19.

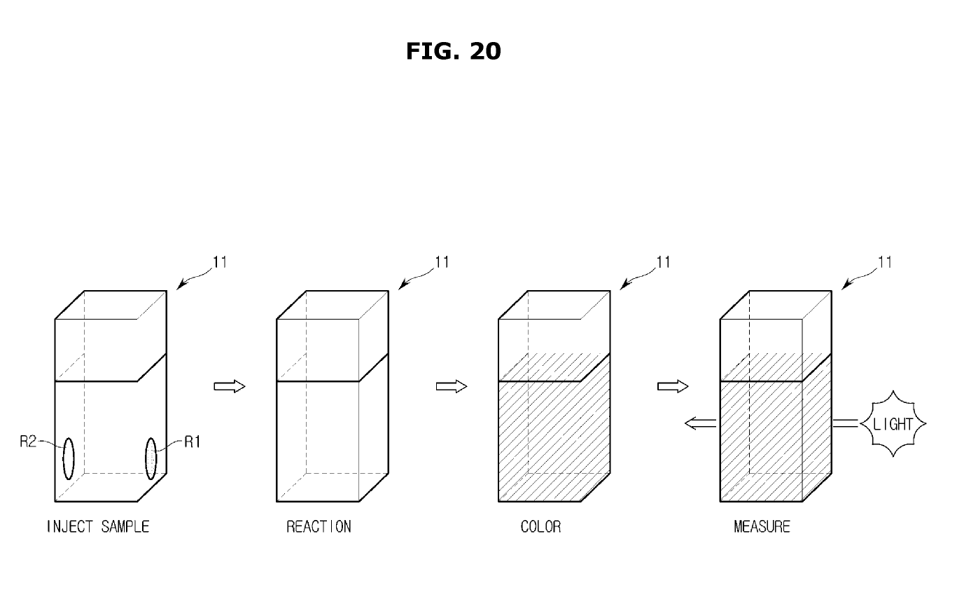

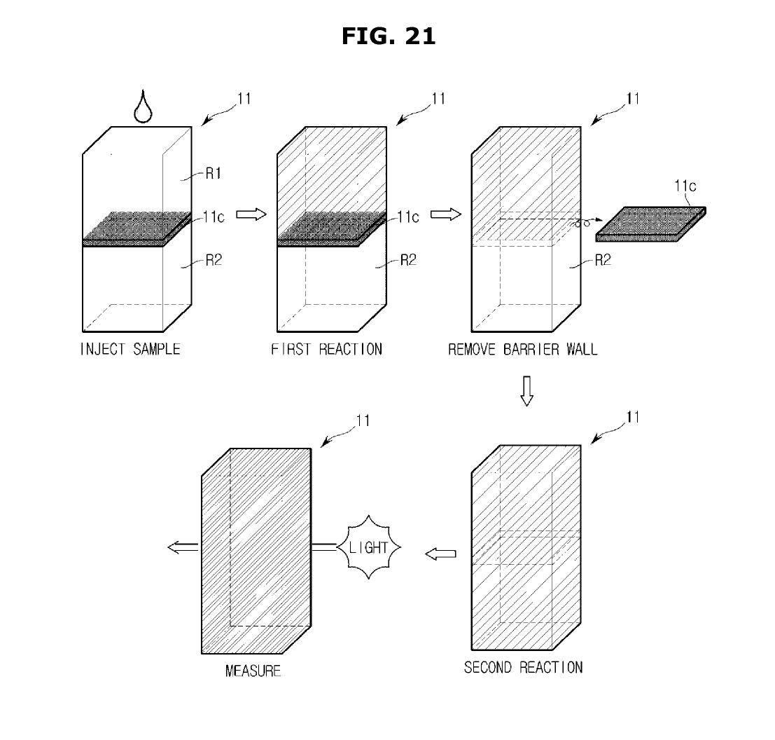

FIGS. 20 and 21 schematically illustrate a reaction that occurs in a sample injected into the microfluidic device of FIGS. 1 and 2.

Referring to FIG. 20, if the sample is injected into the cuvette-shaped chamber 11 in which the first reagent R1 and the second reagent R2 are solidified at inner walls, in accordance with the exemplary embodiment of FIG. 9, the solidified first reagent R1 and the second reagent R2 are dissolved by the sample, and a reaction occurs between the sample and the first reagent R1 and the second reagent R2.

When the first reagent R1 includes antibodies and the second reagent R2 includes an antigen-enzyme conjugant, antigens of the antigen-enzyme conjugant and target antigens included in the sample competitively react with the antibodies of the first reagent R1 within the chamber 11 into which the sample is injected, and a degree of color of the chromogen included in the first reagent R1 or second reagent R2 varies according to the result of the reaction.

In detail, the higher the concentration of the target antigens included in the sample is, the greater the reduction in the number of combinations of the antigens of the antigen-enzyme conjugant and the antibodies, and as the number of combinations of the antigens of the antigen-enzyme conjugant and the antibodies is reduced, the number of specific combinations of the enzymes of the antigen-enzyme conjugant and the temperament increases, and a color reaction increases. If the chamber 11 in which the color reaction occurs is inserted into a spectrometer, optical characteristics, such as OD, transparency, luminous efficiency, or reflectance, can be measured, and the concentration of the target antigens can be estimated from the measured optical characteristics.

Alternatively, when the first reagent R1 and the second reagent R2 are reagents used to detect HbA1c, the sample is injected into the chamber 11, and the sample reacts with the first reagent R1 and the second reagent R2 simultaneously. Thus, if the chamber 11 is injected into the spectrometer, the spectrometer radiates light having a first wavelength so that first OD can be measured, and the spectrometer radiates light having a second wavelength so that second OD can be measured. In particular, the first wavelength may be 570 nm or 535 nm, and the second wavelength may be 660 nm or 630 nm. Thus, the concentration of whole hemoglobin can be estimated from the first OD, and the concentration of HbA1c can be estimated from the second OD.

Referring to FIG. 21, if the sample is injected into the cuvette-shaped chamber 11 in which the first reagent R1 and the second reagent R2 are stored in a liquid state, in accordance with the exemplary embodiment of FIG. 11, the sample and the first reagent R1 meet each other, and a first reaction occurs, and if the barrier wall 11c is removed, the sample and the second reagent R2 meet each other, and a second reaction occurs.

When the first reagent R1 includes antibodies and the second reagent R2 includes an antigen-enzyme conjugant, if the chamber 11 is inserted into the spectrometer, the spectrometer radiates light having a particular wavelength so that OD can be measured. In this aspect, the particular wavelength may be determined according to a type of a chromogen included in the first reagent R1 or second reagent R2.

Alternatively, when the first reagent R1 and the second reagent R2 are reagents used to detect HbA1c, the first reaction may be primary separation caused by protease, and the second reaction may be oxidative cleaving (secondary separation) of fructosylated dipeptides. If the chamber 11 is inserted into the spectrometer, the spectrometer radiates light having a wavelength of 570 nm or 535 nm so that first OD can be measured, and the spectrometer radiates light having a wavelength of 660 nm or 630 nm so that second OD can be measured. The concentration of whole hemoglobin can be estimated from the first OD, and the concentration of HbA1c can be estimated from the second OD. An order of measuring the first OD and the second OD may be reversed.

However, in FIG. 21, the barrier wall 11c is removed after the sample is injected into the chamber 11. However, exemplary embodiments are not limited thereto, and the barrier wall 11 c may also be removed simultaneously with injecting the sample.

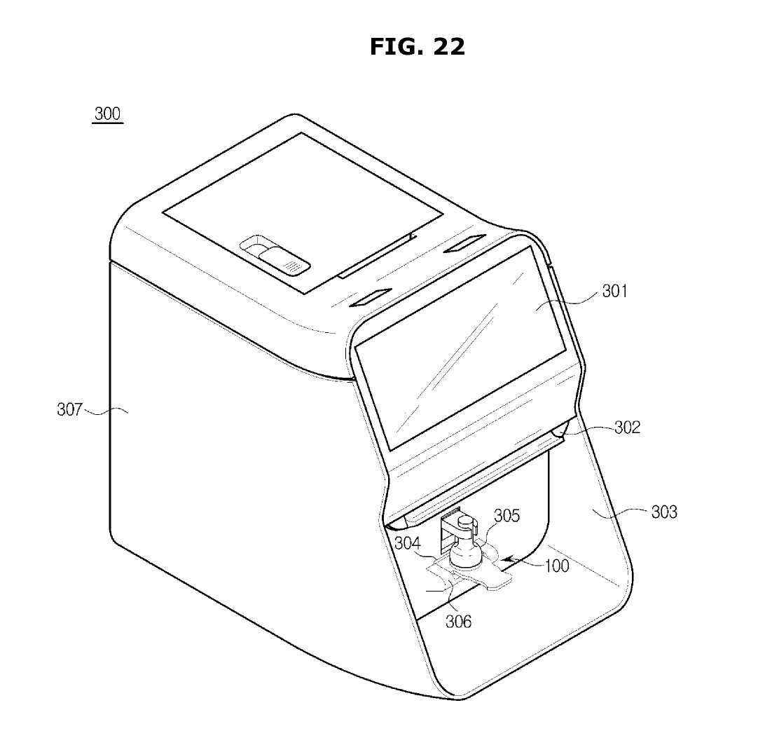

FIG. 22 illustrates an exterior of an apparatus for testing the microfluidic device of FIG. 12.

A testing apparatus 300 is an apparatus for testing the microfluidic device 100 illustrated in FIGS. 12, 13, and 14. Referring to FIG. 22, the testing apparatus 300 includes a mounting portion 303 that is a space in which the microfluidic device 100 is mounted, and if a door 302 of the mounting portion 303 is slid upward and is open, the microfluidic device 100 can be mounted on the testing apparatus 300. As a specific example, the platform 120 of the microfluidic device 100 can be inserted into a predetermined insertion groove 304 which is provided in the mounting portion 303.

The platform 120 may be inserted into a body 307, and the housing 110 may be exposed to an outer side of the testing apparatus 300 and may be supported by a support 306. If a pressurization portion 305 pressurizes the sample supply portion 111, the sample may be prompted to flow into the platform 120.

After the sample flows into the platform 120, the sample is moved to the chamber 12 in which the first reagent R1 and the second reagent R2 are stored, via the channel 122, and the sample simultaneously reacts with the first reagent R1 and the second reagent R2 used to detect HbA1 c within the chamber 12.

If mounting of the microfluidic device 100 is completed, the door 302 is closed, and a test begins. Although not shown, a detector which includes an emission portion (also referred to herein as an “emitter”) and a light receiving portion (also referred to herein as a “light receiver”) is provided in the body 307. The emission portion radiates light having a particular wavelength onto the chamber 12 in which the first reagent R1 and the second reagent R2 are stored, and the light receiving portion detects light that is transmitted from the chamber 12 or is reflected from the chamber 12. When the first reagent R1 and the second reagent R2 are reagents used to detect HbA1c, light having the first wavelength and light having the second wavelength are respectively radiated.

A controller provided at the testing apparatus 300 determines optical characteristics from an output signal of the detector and calculates a concentration of a target material that exists in the sample based on one or more of the optical characteristics.

When the first reagent R1 includes the antibodies and the second reagent R2 includes the antigen-enzyme conjugant, the greater a change in the optical characteristics is, the higher the concentration of the target antigens is. Alternatively, when the first reagent R1 and the second reagent R2 are reagents used to detect a concentration of whole hemoglobin and a concentration of HbA1c that exist in the sample, the concentration of whole hemoglobin that exists in the sample can be calculated from one or more optical characteristics of light having the first wavelength, and the concentration of HbA1c that exists in the sample can be calculated from one or more optical characteristics of light having the second wavelength.

For example, a calibration curve that represents a relationship between OD and the concentration of the target material can be previously stored, and a determined OD can be applied to the calibration curve so that the concentration of the target material can be estimated.

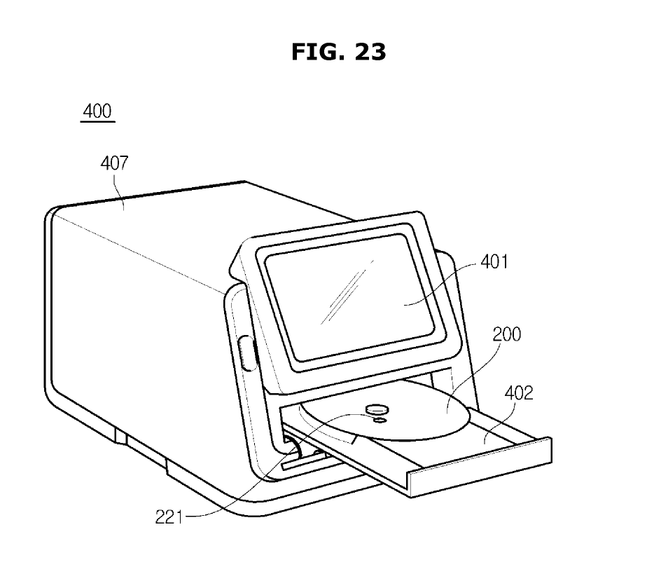

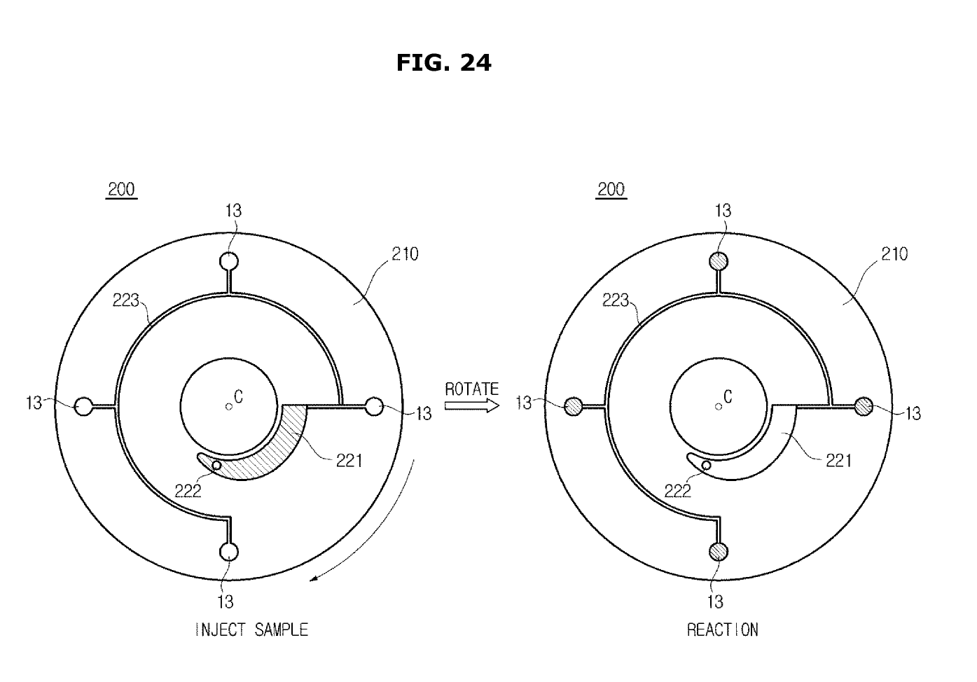

FIG. 23 illustrates an exterior of an apparatus for testing the microfluidic device of FIG. 17, and FIG. 24 illustrates movement of a sample within the microfluidic device mounted on the apparatus for testing the microfluidic device.

Referring to FIGS. 23 and 24, a testing apparatus 400 is used to test the microfluidic device 200 illustrated in FIG. 17. After the sample is injected into the sample accommodation chamber 221 through the sample injection hole 222, the microfluidic device 200 is mounted on a tray 402 of the testing apparatus 400. The mounted microfluidic device 200 is inserted into a body 407 of the testing apparatus 400 together with the tray 402.

If the microfluidic device 200 is inserted into the body 407, the testing apparatus 400 rotates the microfluidic device 200 according to a predetermined sequence, and the sample injected into the sample accommodation chamber 221 is moved to the chamber 13 due to a centrifugal force.

The microfluidic device 200 used in an existing immunity test requires a vibration operation for mixing the reagent and the sample. However, referring to the compositions of the first reagent R1 and the second reagent R2 shown in Tables 1 and 2, the first reagent R1 and the second reagent R2 included in the chamber 13 are formed of materials which have excellent solubility with respect to the sample. Thus, since no additional vibration operation is required, a testing time can be reduced.

Although not shown, a detector including an emission portion (also referred to herein as an “emitter”) and a light receiving portion (also referred to herein as a “light receiver”) is provided in the body 407. The emission portion radiates light having a particular wavelength onto the chamber 13 in which the first reagent R1 and the second reagent R2 are stored, and the light receiving portion detects light that is transmitted from the chamber 13 or that is reflected from the chamber 13.

Similarly as in the above-described testing apparatus 300, when the first reagent R1 and the second reagent R2 are reagents used to detect hemoglobin and HbA1c, light having the first wavelength and light having the second wavelength are respectively radiated. The testing apparatus 400 can determine OD from a signal output by the light receiving portion and can estimate the concentration of the target material based on the determined OD.

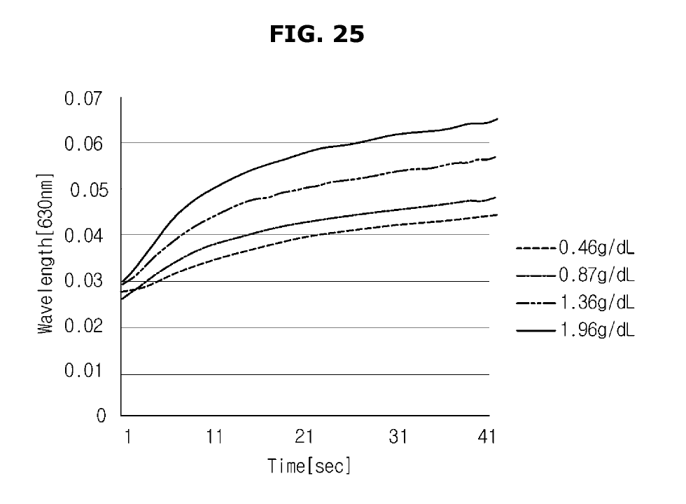

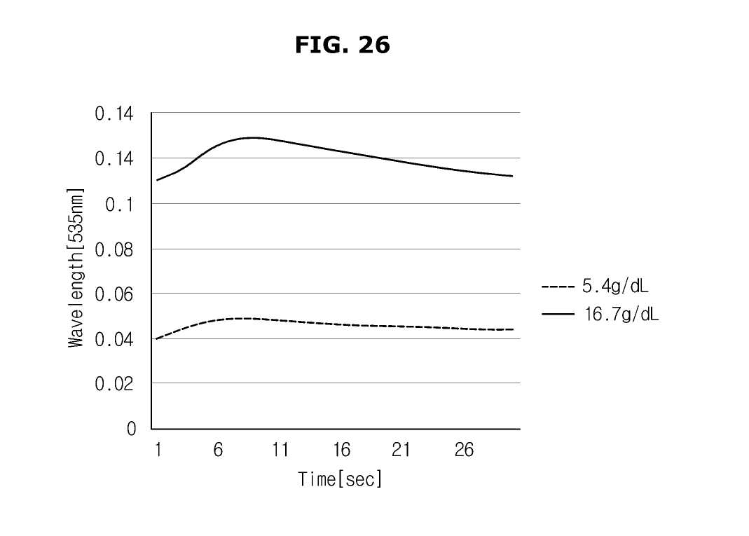

FIG. 25 is a graph showing optical density (OD) obtained by radiating light having a wavelength of 630 nm onto a chamber in which a reagent for detecting glycated hemoglobin is stored, and FIG. 26 is a graph showing OD obtained by radiating light having a wavelength of 535 nm onto the same chamber.

In the current experiments, after samples including 0.46 g/dL, 0.87 g/dL, 1.36 g/dL, and 1.96 g/dL of HbA1c were respectively injected into the chamber 10 in which the first reagent R1 and the second reagent R2 used to detect whole hemoglobin and HbA1c were stored, light having a wavelength of 630 nm was radiated, and light that was transmitted from the chamber 10 was detected such that ODs were obtained. Thus, a result thereof is shown in a graph of FIG. 25. In particular, an optical path formed by the chamber 10 was 0.16 mm.

At least one of the samples injected into the chamber 10 includes 5.4 g/dL of whole hemoglobin, and at least another one thereof includes 16.7 g/dL of whole hemoglobin. After OD with respect to light having a wavelength of 610 nm was measured, light radiated onto the chamber 10 was changed to have a wavelength of 535 nm, and a result of measuring OD is shown in a graph of FIG. 26.

Referring to FIGS. 25 and 26, as both a concentration of whole hemoglobin and a concentration of HbA1c increased, OD increased. Thus, when both the first reagent R1 and the second reagent R2 simultaneously reacted with the sample in one chamber 10, and a wavelength was changed such that ODs were measured, a result in which discrimination between concentrations was recognized could be obtained.

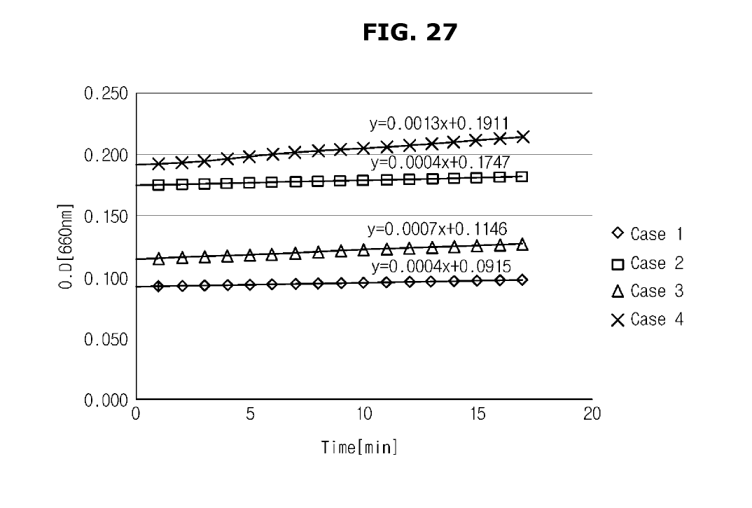

FIG. 27 is a graph showing a result of measuring OD by varying concentration of enzymes included in the reagent with respect to a sample including glycated hemoglobin having the same concentrations.

In the current experiments, concentrations of enzymes included in the first reagent R1 and the second reagent R2 were different from each other with respect to the sample including 0.46 g/dL of HbA1c such that ODs were measured, and an optical path formed by the chamber 10 was less than or equal to 0.1 mm.

In Case 1, a concentration of FPDX included in the first reagent R1 was 600 KU/L, and a concentration of POD was 25 KU/L, and a concentration of thermolysin included in the second reagent R2 was 400 KU/L such that ODs were measured.

In Case 2, a concentration of FPDX included in the first reagent R1 was 600 KU/L, and a concentration of POD was 25 KU/L, and a concentration of thermolysin included in the second reagent R2 was 4000 KU/L such that ODs were measured.

In Case 3, a concentration of FPDX included in the first reagent R1 was 6000 KU/L, and a concentration of POD was 250 KU/L, and a concentration of thermolysin included in the second reagent R2 was 400 KU/L such that ODs were measured.

In Case 4, a concentration of FPDX included in the first reagent R1 was 6000 KU/L, and a concentration of POD was 250 KU/L, and a concentration of thermolysin was 4000 KU/L such that ODs were measured.

Referring to FIG. 27, in Case 4 in which a concentration of FPDX was 6000 KU/L and a concentration of thermolysin was 4000 KU/L, a gradient of OD over time is the largest, and next, in Case 2 in which a concentration of FPDX was 600 KU/L, a concentration of POD was 25 KU/L, and a concentration of thermolysin was 4000 KU/L, a gradient of OD over time is also relatively large.

The higher the concentration of enzymes included in the reagent is, the more a change in ODs over time increases. Thus, the concentration of the target material can be estimated from a corresponding amount in a change of ODs. Thus, the higher the concentration of enzymes included in the reagent is, the more the concentration discrimination increases from the result of FIG. 27.

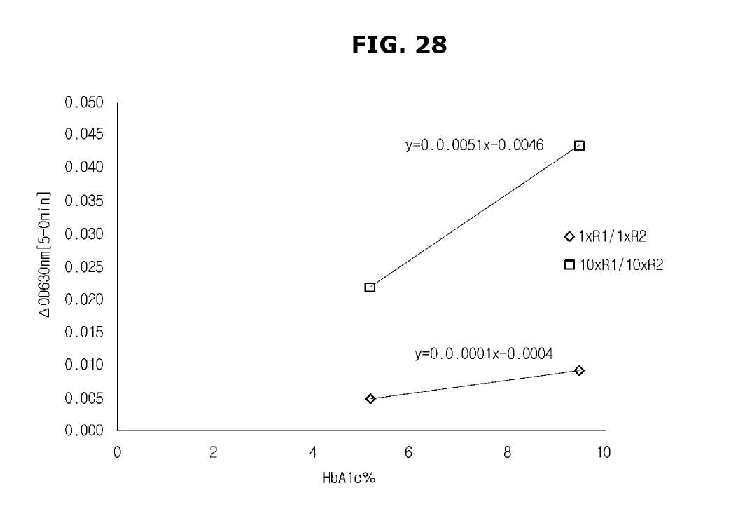

FIG. 28 is a graph showing a result of measuring OD by increasing a concentration of enzymes by a factor of ten with respect to a sample having glycated hemoglobin having different concentrations.

In the current experiments, concentrations of enzymes with respect to two samples having a control level 1 (HbA1c %=5.2) of HbA1c and a control level 2 (HbA1c %=9.5) of HbA1c were increased by a factor of ten such that ODs were measured. An optical path formed by the chamber 10 was less than or equal to 0.1 mm.

Referring to FIG. 28, as the concentrations of the enzymes increased by a factor of ten, a difference in ODs between the control level 1 and the control level 2 increased rapidly. Thus, as the concentrations of the enzymes increased, discrimination between the concentrations increased.

Further, in the above experiments, the optical path was set to be less than or equal to 0.1 mm. Thus, when the optical path is reduced, there is a high probability that precision and/or accuracy of a testing result will be reduced. However, as shown in the graph of FIG. 27 and the graph of FIG. 28, the concentrations of the enzymes included in the reagent were increased such that discrimination between the concentrations was improved. Thus, a microfluidic device in accordance with an exemplary embodiment was implemented to have a small thickness, and simultaneously, the concentrations of the enzymes included in the reagent were increased, so that demand for miniaturization of the microfluidic device and improvements in performance of the microfluidic device could be simultaneously satisfied.

Hereinafter, a result of testing a performance of the microfluidic device in accordance with an exemplary embodiment will be described.

The following Table 3 shows a result of testing precision of the microfluidic device in accordance with an exemplary embodiment.

TABLE 3

| Control | % HbA1c | SD | CV[%] |
|---|---|---|---|
| Low | 5.2 | 0.173 | 3.3 |
| High | 9.5 | 0.337 | 3.5 |

Precision is an index that represents how reproducibility of a device is excellent. Precision may be expressed using a standard deviation (SD) and a coefficient variation (CV), and the coefficient variation (CV) represents as a percentage of the standard deviation (SD) with respect to the mean.

In the current test, the SD and the CV were calculated by measurement 20 times (n=20). Since the lower the CV is, the higher precision is, a device having a CV of 5% or less has excellent reproducibility.

Referring to Table 3 above, since the CV in the range of 3% was calculated at both a high concentration and a low concentration, the microfluidic device in accordance with an exemplary embodiment has excellent reproducibility.

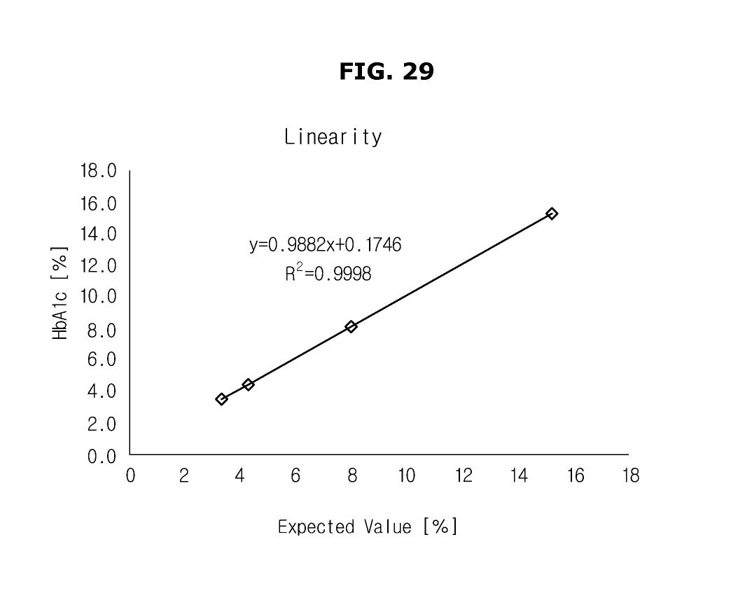

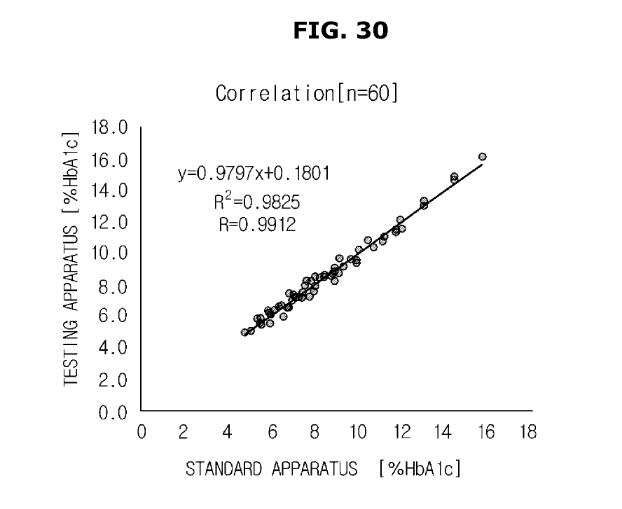

FIG. 29 is a graph showing linearity of a result of testing by a microfluidic device, in accordance with an exemplary embodiment, and FIG. 30 is a graph showing correlation of a result of testing a microfluidic device, in accordance with an exemplary embodiment.

Linearity represents a degree to which an actual concentration value (prediction value) of a target material and a measured value form a straight line. This also represents a range in which a measurement value obtained by the device may be reliable. Thus, the measurement value can be reliable with respect to a section in which linearity is maintained.

Referring to the result of FIG. 29, since linearity is maintained in a section in which the concentration of HbA1c is within a range of 3% to 16%, when a measurement value obtained by the microfluidic device in accordance with an exemplary embodiment is in the range of 3 to 16%, the measurement value can be reliable.

Correlation is an index that represents correlation of a testing result between a device which corresponds to a standard and a device for which performance is to be evaluated, and accuracy can be indirectly evaluated by correlation. Correlation may be represented as a correlation coefficient R, and as an absolute value of the correlation coefficient R is closer to one (i.e., 1.00), accuracy may be high.

In the current test, the microfluidic device 100 illustrated in FIGS. 12, 13, and 14 was inserted into the testing apparatus 300 illustrated in FIG. 22 so as to measure the concentration of HbA1c that existed in 60 clinical samples (n=60), and specimens in the same condition were injected into a large clinical chemistry automatic analysis device, so that a correlation between two results was measured.

Referring to FIG. 30, the correlation coefficient R was calculated as 0.9912. Since this value is close to 1, an accuracy of the microfluidic device in accordance with an exemplary embodiment may also be high.

In the microfluidic device and the apparatus for testing the same described above, a reaction required for an in vitro diagnosis, such as an immunity test and a clinical chemistry test, occurs in one chamber so that miniaturization of the device can be implemented, and the reaction occurs in one chamber in one step so that a rapid test can be performed.

As described above, in a microfluidic device according to one or more exemplary embodiments, an in vitro diagnosis can be rapidly performed, and the microfluidic device can be miniaturized.

Although a few exemplary embodiments have been shown and described, it will be appreciated by those skilled in the art that changes may be made in these exemplary embodiments without departing from the principles and spirit of the present inventive concept, the scope of which is defined in the claims and their equivalents.

What is claimed is:

1. A microfluidic device comprising:
a platform which includes a sample injection hole through which a sample is injectable; and
a chamber which is formed in the platform and which is configured to store a first reagent, which includes target antigens that exist in the sample and antibodies that are specifically combined with the target antigens, and a second reagent, which includes an antigen-enzyme conjugant in which antigens that are specifically combined with the antibodies and enzymes are conjugated.

2. The microfluidic device of claim 1, wherein the target antigens that exist in the sample and the antigen-enzyme conjugant included in the second reagent are competitively combined with the antibodies included in the first reagent.

3. The microfluidic device of claim 2, wherein at least one from among the first reagent and the second reagent comprises a temperament that is specifically combined with the enzymes of the antigen-enzyme conjugant.

4. The microfluidic device of claim 3, wherein the at least one from among the first reagent and the second reagent further comprises a chromogen of which a degree of color varies based on an amount of the temperament that is specifically combined with the enzymes of the antigen-enzyme conjugant.

5. The microfluidic device of claim 4, wherein the platform comprises a film-shaped upper plate and a film-shaped lower plate, and
the chamber is formed by bonding the upper plate with the lower plate.

6. The microfluidic device of claim 5, wherein the first reagent is applied onto a first one of the upper plate and the lower plate and then is dried, and
the second reagent is applied onto an other one of the upper plate and the lower plate and then is dried.

7. The microfluidic device of claim 5, further comprising a channel that is formed at the platform and which is configured to connect the sample injection hole with the chamber.

8. The microfluidic device of claim 7, further comprising a filter that is disposed at the sample injection hole and which is configured to filter a particular material included in the sample.

9. The microfluidic device of claim 4, further comprising a sample accommodation chamber that is formed at the platform and which is configured to accommodate the sample injected through the sample injection hole.

10. The microfluidic device of claim 9, wherein the platform is rotatable, and the sample accommodation chamber is disposed closer to a center of rotation of the platform than the chamber.

11. The microfluidic device of claim 10, wherein the first reagent is applied at a first position of inner walls of the chamber and then dried, and the second reagent is applied at a second position of the inner walls of the chamber and then dried, wherein the second position is different than the first position.

12. The microfluidic device of claim 10, wherein the first reagent and the second reagent are stored in the chamber in a solid state.

13. The microfluidic device of claim 10, further comprising a channel configured to connect the chamber with the sample accommodation chamber.

14. The microfluidic device of claim 4, wherein the first reagent and the second reagent are stored in the chamber in a liquid state, and the chamber comprises a barrier wall that separates a first space in which the first reagent is stored from a second space in which the second reagent is stored.

15. A microfluidic device comprising:
a platform which includes a sample injection hole through which a sample is injectable; and
a chamber which is formed in the platform and which is configured to store a first reagent, which includes first enzymes that primarily decompose hemoglobin that exists in the sample, and a second reagent, which includes second enzymes that secondarily decompose the decomposed hemoglobin.

16. The microfluidic device of claim 15, wherein the first enzymes that primarily decompose the hemoglobin are protease-based, and
the second enzymes that secondarily decompose the decomposed hemoglobin are fructosyl-based.

17. The microfluidic device of claim 15, wherein the platform comprises a film-shaped upper plate and a film-shaped lower plate, and
the chamber is formed by bonding the upper plate with the lower plate.

18. The microfluidic device of claim 17, wherein the first reagent is applied onto a first one of the upper plate and the lower plate and then is dried, and
the second reagent is applied onto an other one of the upper plate and the lower plate and then is dried.

19. The microfluidic device of claim 15, further comprising a sample accommodation chamber that is formed at the platform and which is configured to accommodate the sample injected through the sample injection hole,
wherein the platform is rotatable, and
the sample accommodation chamber is disposed closer to a center of rotation of the platform than the chamber.

20. The microfluidic device of claim 18, wherein the first reagent is applied at a first position of inner walls of the chamber and then dried, and the second reagent is applied at a second position of the inner walls of the chamber and then dried, wherein the second position is different from the first position.

21. The microfluidic device of claim 15, wherein the first reagent and the second reagent are stored in the chamber in a solid state.

22. The microfluidic device of claim 15, wherein the first reagent and the second reagent are stored in the chamber in a liquid state, and
the chamber comprises a barrier wall that separates a first space in which the first reagent is stored from a second space in which the second reagent is stored.

23. An apparatus for testing the microfluidic device of claim 4, comprising:
a detector configured to radiate light having a particular wavelength onto the chamber and to detect light that is transmitted from the chamber or is reflected from the chamber; and
a controller configured to determine a change in at least one from among a plurality of optical characteristics from an output signal of the detector and to calculate a respective increase in a concentration of the target antigens which corresponds to an increase in the change in the at least one of the plurality of optical characteristics.

24. An apparatus for testing the microfluidic device of claim 15, comprising:
a detector configured to radiate first light having a first wavelength onto the chamber and to detect second light that is transmitted from the chamber or is reflected from the chamber, and to radiate third light having a second wavelength that is different from the first wavelength onto the chamber and to detect fourth light that is transmitted from the chamber or is reflected from the chamber; and
a controller configured to calculate a concentration of hemoglobin that exists in the sample from at least one from among a plurality of optical characteristics of the first light having the first wavelength and to calculate a concentration of glycated hemoglobin that exists in the sample from at least one from among a plurality of optical characteristics of the third light having the second wavelength.

25. The apparatus of claim 24, wherein the first wavelength is a wavelength in a band of 500 nm, and the second wavelength is a wavelength in a band of 600 nm.
